(19) United States
(12) Patent Application Publication
(10) Pub. No.: US 2006/0206211 A1
(43) Pub. Date: Sep. 14, 2006

(54) Bipolar hip prosthesis with free floating ring

(75) Inventors: Michael Daniels, Warsaw, IN (US); Nathaniel Young, Draper, UT (US)

Correspondence Address: STOEL RIVES LLP - SLC, 201 SOUTH MAIN STREET, ONE UTAH CENTER, SALT LAKE CITY, UT, 84111, US

(73) Assignee: Ortho Development Corporation

(21) Appl. No.: 11/343,470

(22) Filed: Jan. 30, 2006

Related U.S. Application Data
(60) Provisional application No. 60/655,285, filed on Feb. 22, 2005.

(51) Int. Cl. A61F 2/32

(52) U.S. Cl. 623/22.17; 623/22.24

(57) ABSTRACT

A bipolar hip prosthesis is disclosed. The bipolar prosthesis generally includes an acetabular shell component that may articulate within the acetabulum of the patient and that may be configured and dimensioned to receive a bearing component therein. The bearing component may be configured and dimensioned for receiving a prosthetic femoral head component therein, wherein the prosthetic femoral head component may articulate. The bearing component may further comprise a recess to house a uniquely designed free floating ring to capture the prosthetic femoral head component therein.

### CROSS-REFERENCE TO RELATED APPLICATIONS

This application claims the benefit of U.S. Provisional Application No. 60/655,285, filed Feb. 22, 2005, which is hereby incorporated by reference herein in its entirety, including but not limited to those portions that specifically appear hereinafter, the incorporation by reference being made with the following exception: In the event that any portion of the above-referenced provisional application is inconsistent with this application, this application supercedes said above-referenced provisional application.

### STATEMENT REGARDING FEDERALLY SPONSORED RESEARCH OR DEVELOPMENT

Not Applicable.

### BACKGROUND

1. The Field of the Invention

The present disclosure relates generally to prostheses used in hip surgical procedures, and more particularly, but not necessarily entirely, to the features that mechanically interconnect a femoral head component within a bipolar prosthesis forming at least a part of a hip joint.

2. Description of Related Art

The human hip joint acts mechanically as a ball and socket joint, wherein the ball-shaped head of the natural femur is positioned within the natural socket-shaped acetabulum of the pelvis. In a total hip joint replacement, both the natural femoral head and the natural surface of the acetabulum are replaced with prosthetic devices. A total hip replacement is typically used when both the natural femoral head and natural acetabulum are diseased or damaged. Often, however, only the natural femoral head of a patient is diseased or damaged. In such circumstances, in order to be bone sparing which is advantageous in hip surgical procedures, a hip hemiarthroplasty surgical procedure is commonly used. In a hip hemiarthroplasty surgical procedure, the proximal portion of a femur is replaced with a suitable prosthetic implant that articulates directly with a patient's natural acetabulum.

In a hip hemiarthroplasty procedure, two types of femoral prostheses are typically used. One type is a bipolar prosthesis. In general, a bipolar hip prosthesis includes an acetabular shell component having an external surface that articulates with a patient's natural acetabulum. The bipolar prosthesis also includes a bearing component that comprises an internal surface that articulates with a spherical head member of a prosthetic femoral component.

The other type of prosthesis is often referred to as a unipolar endoprosthesis in which the prosthetic femoral component includes a spherical head member that is large enough to articulate directly with the natural acetabulum.

The present disclosure relates to a bipolar hip prosthesis. In the prior art, there are many femoral head components and many acetabular shell components that can be used together to create a bipolar hip joint. During surgery, the femoral head component may be implanted into the femur in a first procedure. The acetabular shell component may be inserted into the natural acetabulum in a second procedure. The femoral head component may then be joined to the acetabular shell component in a third procedure.

One concern of all such prior art devices is how to set the femoral head component in the bearing component of the bipolar prosthesis so that the femoral head component does not become displaced from the bearing component during normal activity. It is a great complication when a bipolar hip prosthesis becomes displaced after insertion. In a displacement, the spherical head of the femoral component becomes dissociated from the bearing component. Such a dissociation most typically occurs as the result of an abnormal twisting of the leg or after a trauma, such as a fall.

In the prior art, various locking mechanisms have been used to lock the femoral head component in place within the bearing component of the bipolar prosthesis and prevent the femoral head component from becoming displaced. Such prior art is exemplified by U.S. Pat. No. 4,798,610 to Averill et al., entitled Prosthetic Implant Device. The Averill et al. patent is itself an improvement of U.S. Pat. No. 4,241,463 (Khovaylo). In the Averill et al. patent, the femoral head component is mechanically prevented from dislocating from the bearing component by a free floating locking ring. The bearing component comprises a recess that is partially defined by a circumferential lip, wherein the ring is located within the recess and contacts the lip thereby preventing dislocation of the femoral head component from the bearing component. The ring itself comprises an inner surface that has an arcuate cross-sectional shape for contacting the spherical femoral head component when that component is urged downwardly against the ring by downward forces tending to disengage the femoral head component from the bearing component.

In order to solve the problems associated with displacement of the femoral head component from the bearing component of the bipolar prosthesis, devices have been developed that allow a displaced femoral head component to be reset into a bearing component without intrusive surgery. One such bipolar hip prosthesis is shown in U.S. Pat. No. 6,206,929 to Ochoa et al., entitled Bipolar Hip Prosthesis With Locking Head. In the Ochoa et al. patent, flexible locking elements hold the femoral head component within the bearing and shell components. During trauma, the femoral head component may be pulled out of the bipolar prosthesis. However, the femoral head component can be set back into the bearing component of the bipolar prosthesis without surgery, provided the flexible locking elements were not damaged by the removal of the femoral head component. Unfortunately, during different traumas, damage may sometimes occur to the locking elements. If damage to the locking elements does occur, then intrusive surgery may be required to either replace various parts of the bipolar prosthesis, such as the bearing component, or to assemble the acetabular shell component and the bearing component back onto the femoral head component.

The features and advantages of the disclosure will be set forth in the description which follows, and in part will be apparent from the description, or may be learned by the practice of the disclosure without undue experimentation. The features and advantages of the disclosure may be realized and obtained by means of the instruments and combinations particularly pointed out in the appended claims.

### BRIEF DESCRIPTION OF THE DRAWINGS

The features and advantages of the disclosure will become apparent from a consideration of the subsequent detailed description presented in connection with the accompanying drawings in which:

FIG. 1 is a schematic view of a bipolar hip prosthesis including a shell component, a bearing component, a free floating ring, and a femoral component all made in accordance with the principles of the present disclosure;

FIG. 2 is a perspective view of a bearing component of a bipolar hip prosthesis made in accordance with the principles of the present disclosure;

FIG. 3 is a side, cross-sectional view of the bearing component of the bipolar hip prosthesis of FIG. 2, taken along section A-A;

FIG. 4 is a bottom view of an alternative embodiment of a bearing component of a bipolar hip prosthesis made in accordance with the principles of the present disclosure;

FIG. 5 is a side view of the bearing component of FIG. 4;

FIG. 6 is a side, cross-sectional view of the bearing component of the bipolar hip prosthesis of FIG. 4, taken along section A-A;

FIG. 7 is an enlarged, detail view of a recess formed in the bearing component of the bipolar hip prosthesis of FIG. 6;

FIG. 8 is a side view of a free floating ring of the bipolar hip prosthesis made in accordance with the principles of the present disclosure;

FIG. 9 is a top view of the free floating ring of the bipolar hip prosthesis of FIG. 8;

FIG. 10 is an enlarged side, cross-sectional view of a portion of the free floating ring of the bipolar hip prosthesis of FIG. 9, taken along section A-A;

FIG. 11 is a top view of an alternative embodiment of the free floating ring of the bipolar hip prosthesis made in accordance with the principles of the present disclosure;

FIG. 12 is a side, cross-sectional view of the free floating ring of the bipolar hip prosthesis of FIG. 11, taken along section A-A;

FIG. 13 is an enlarged, detail view of a portion of the free floating ring of the bipolar hip prosthesis of FIG. 12;

FIG. 14 is an enlarged side, cross-sectional view of a portion of the free floating ring of the bipolar hip prosthesis of FIG. 9, illustrating a point of convergence of an outer surface and inner surface of the ring; and

FIG. 15 is an enlarged, detail view of the free floating ring of the bipolar hip prosthesis of FIG. 12, illustrating a point of convergence of an outer surface and inner surface of the ring.

### DETAILED DESCRIPTION

For the purposes of promoting an understanding of the principles in accordance with the disclosure, reference will now be made to the embodiments illustrated in the drawings and specific language will be used to describe the same. It will nevertheless be understood that no limitation of the scope of the disclosure is thereby intended. Any alterations and further modifications of the inventive features illustrated herein, and any additional applications of the principles of the disclosure as illustrated herein, which would normally occur to one skilled in the relevant art and having possession of this disclosure, are to be considered within the scope of the disclosure claimed.

It is to be understood that this disclosure is not limited to the particular configurations, process steps, and materials disclosed herein as such configurations, process steps, and materials may vary somewhat. It is also to be understood that the terminology employed herein is used for the purpose of describing particular embodiments only and is not intended to be limiting since the scope of the present disclosure will be limited only by the appended claims and equivalents thereof.

The publications and other reference materials referred to herein to describe the background of the disclosure, and to provide additional detail regarding its practice, are hereby incorporated by reference herein in their entireties, with the following exception: In the event that any portion of said reference materials is inconsistent with this application, this application supercedes said reference materials. The reference materials discussed herein are provided solely for their disclosure prior to the filing date of the present application. Nothing herein is to be construed as a suggestion or admission that the inventors are not entitled to antedate such disclosure by virtue of prior disclosure, or to distinguish the present disclosure from the subject matter disclosed in the reference materials.

It must be noted that, as used in this specification and the appended claims, the singular forms “a,” “an,” and “the” include plural referents unless the context clearly dictates otherwise.

In describing and claiming the subject matter of the present disclosure, the following terminology will be used in accordance with the definitions set out below.

As used herein, the terms “comprising,” “including,” “containing,” “characterized by,” and grammatical equivalents thereof are inclusive or open-ended terms that do not exclude additional, unrecited elements or method steps.

As used herein, the term “proximal” shall refer broadly to the concept of a nearest portion. For example, “proximal” may anatomically refer to an area or point of reference that is nearer another point, such as an origin, a point of attachment, or the midline of the body.

As used herein, the term “distal” shall refer broadly to the concept of a furthest portion, or shall generally refer to the opposite of proximal. For example, “distal” may anatomically refer to an area or point of reference that is located far from another point of reference, such as an origin, a point of attachment, or the midline of the body.

Applicants have discovered that the effectiveness of a bipolar hip prosthesis may be increased by utilizing the concepts and principles set forth in the present disclosure, and particularly by utilizing a uniquely designed free floating ring in conjunction with a modular bipolar prosthesis comprised of an acetabular shell component and a bearing component. Additionally, the efficiency in inserting a prosthetic femoral head component into a corresponding bearing component of the bipolar prosthesis may be enhanced by utilizing the uniquely designed free floating ring and bearing component of the present disclosure.

It should be noted that in the figures similar features and elements of a bipolar hip prosthesis of the present disclosure will be identified using the same reference numerals in the various embodiments with the only exception being the first digit of the two or three digit reference numeral indicating a different embodiment.

Further, it should be noted that any dimensions contained in the figures are for exemplary purposes only, and are in no way intended to limit the scope of the present disclosure. Rather, such dimensions have been retained in the drawings for purposes of disclosure and illustration only. One of ordinary skill in the art will appreciate that the dimensions contained in the figures may be changed or modified without departing from the spirit or scope of the principles of the present disclosure.

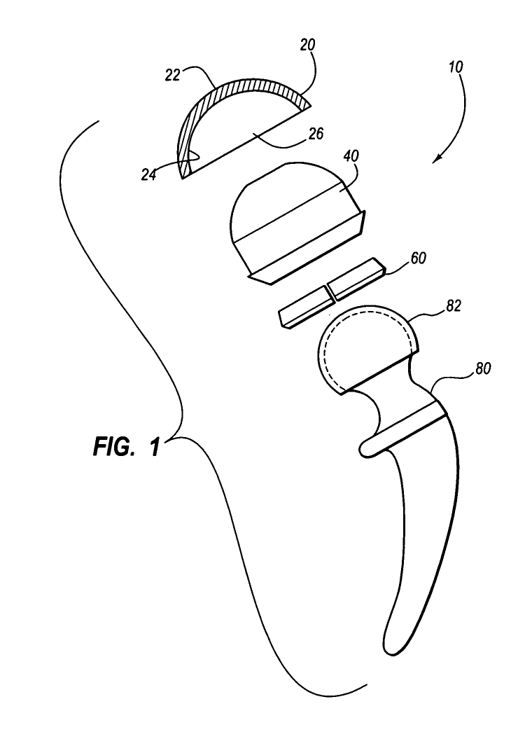

Referring specifically to FIG. 1, a bipolar hip prosthesis 10 of the present disclosure is illustrated, and may comprise an acetabular shell component 20, a bearing or liner component 40, a free floating ring component 60, and a femoral component 80.

The acetabular shell component 20 may be a substantially semi-spherical member and may comprise an outer surface 22 and an inner surface 24. It will be appreciated that the shell component 20 may be configured and dimensioned to articulate with the natural acetabulum of the hip bone. More specifically, the outer surface 22 of the shell component 20 may be configured and dimensioned to articulate directly with the acetabulum of the hip bone. Such articulation between the outer surface 22 of the shell component 20 and the acetabulum of the hip bone may constitute the first articulation of the bipolar prosthesis 10.

The inner surface 24 of the shell component 20 may be dome shaped and may define an inner semi-spherical cavity 26 for receiving the bearing component 40 therein.

It will be appreciated that the shell component 20 may be manufactured from any suitable biocompatible material, including metal, such as titanium, stainless steel, cobalt-chromium-molybdenum alloy, titanium-aluminum vanadium alloy or other suitable metallic alloys, or non-metallic biocompatible materials such as carbon-fiber, ceramic, bio-resorbable materials or if desired any suitable high strength plastic such as an ultra high molecular weight polyethylene that is capable of functioning as an articulating surface with the acetabulum. It will be appreciated by those skilled in the art that other biocompatible materials, whether now known or later discovered, may be utilized by the present disclosure, and said biocompatible materials are intended to fall within the scope of the present disclosure.

It will be appreciated that the shell component 20 of the bipolar prosthesis 10 of the present disclosure may be utilized with many different bearing components 40, whether disclosed herein or otherwise without departing from the scope of the present disclosure. It will be appreciated that the features of the bearing component 20 of the present disclosure will be discussed herein below.

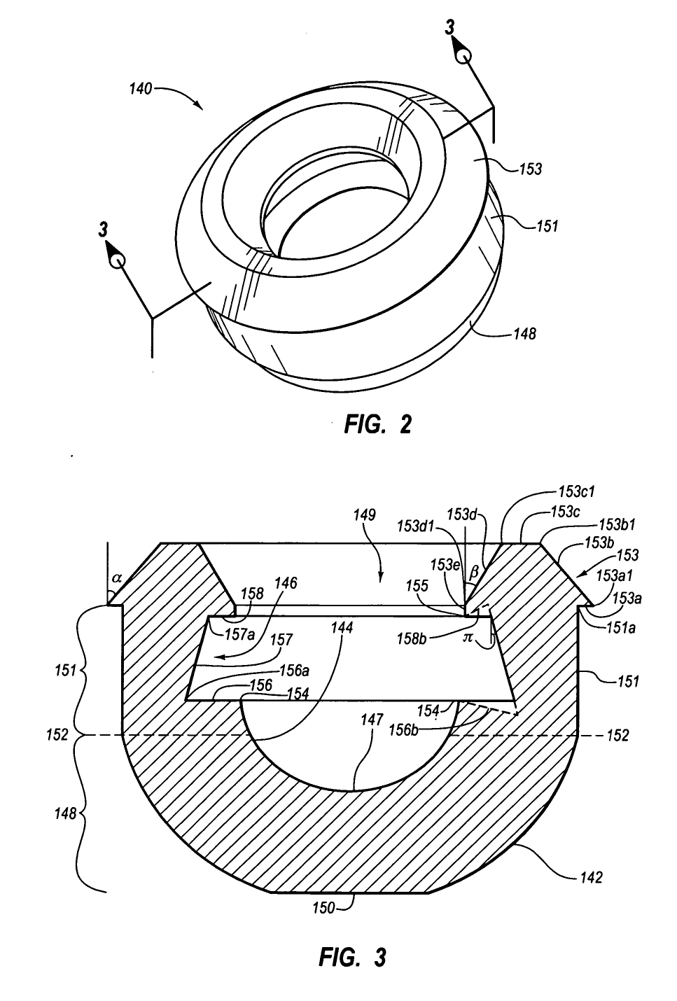

Referring now to FIGS. 2 and 3, an embodiment of a bearing component 140 of the present disclosure is illustrated and may comprise an outer surface 142, an inner surface 144, a recess 146, a bearing cavity 147 and an opening 149 into the bearing cavity 147. It will be appreciated that the bearing component 140 may be configured and dimensioned to seat within the interior cavity 26 of the shell component 20 and may be attached thereto by a means for attaching the bearing component 140 to the shell component 20. Such means for attaching are readily known in the prior art and may include all such prior art mechanisms for attaching the bearing component 140 to the shell component 20.

Each of the bearing component embodiments of the present disclosure may be manufactured from various biocompatible materials, including biocompatible polymeric materials such as polyethylene or ultra high molecular weight polyethylene, or biocompatible ceramic materials, or even biocompatible metallic materials without departing from the scope of the present disclosure.

It will be appreciated that the outer surface 142 of the bearing component 140 may form a substantially semi-spherical dome portion 148 that may extend from approximately an apex region 150 of the bearing component 140 to approximately a midline 152 of the bearing component 140. From the midline 152, the outer surface 142 may comprise a cylindrical surface 151 that may extend toward the opening 149 of the bearing cavity 147 and terminate at a rim 153 of the bearing component 140. The rim 153 of the bearing component 140 may circumferentially extend around the perimeter of the bearing component 140.

It will be appreciated that the shape of the rim 153 may comprise various shapes and forms. However, the rim 153 illustrated in FIG. 3 may be defined by a first laterally extending wall 153a, a first tapered wall 153b, a second laterally extending wall 153c, a second tapered wall 153d, and a first longitudinal wall 153e.

It is to be understood that the rim 153 may be defined with reference to the bearing component 140 oriented in the manner illustrated in FIG. 3. When the bearing component 140 is oriented as such, the first laterally extending wall 153a may extend outwardly from a terminal end 151a of the cylindrical surface 151 in a lateral manner away from the inner surface 144 of the bearing component 140. It will be appreciated that the first laterally extending wall 153a may extend at substantially a ninety degree (90°) angle with respect to a longitudinal axis A-A of the bearing component 140. However, one of ordinary skill in the art will appreciate that such an angle may be modified without departing from the spirit and scope of the present disclosure.

With the bearing component 140 positioned in the orientation of FIG. 3, the rim 153 may be further defined by the first tapered wall 153b. The wall 153b may taper upwardly and inwardly, or in other words may taper inwardly in a proximal to distal direction with respect to the midline 152, at an angle a from a terminal end 153a1 of the first laterally extending wall 153a. The first tapered wall 153b may comprise an angle α with respect to a line parallel to the longitudinal axis A-A of the bearing component 140 that may be between the range of about fifteen degrees to about sixty-five degrees (15°-65°), specifically between about thirty degrees and about fifty-five degrees (30°-55°), and more specifically between about forty degrees to about fifty degrees (40°-50°), and even more specifically about forty-five degrees (45°).

The second laterally extending wall 153c may extend inwardly from a terminal end 153b1 of the first tapered wall 153b in a substantial lateral direction toward the inner surface 144 of the bearing component 140.

The second tapered wall 153d may extend downwardly and inwardly, or in other words may taper inwardly in a distal to proximal direction with respect to the midline 152, at an angle β from a terminal end 153c1 of the second laterally extending wall 153c. The second tapered wall 153d may comprise an angle β with respect to a line parallel to the longitudinal axis A-A of the bearing component 140 that may be between the range of about ten degrees to about fifty degrees (10°-50°), specifically between about twenty degrees and about forty degrees (20°-40°), and more specifically between about twenty-five degrees to about thirty-five degrees (25°-35°), and even more specifically about thirty degrees (30°).

Finally, the first longitudinal wall 153e may extend downwardly from a terminal end 153d1 of the second tapered wall 153d in a substantial longitudinal direction defining the remainder of the rim 153.

The inner surface 144 of the bearing component 140 may define the bearing cavity 147. As illustrated best in FIG. 3, the bearing cavity 147 may be substantially dome shaped or semi-spherical in nature, such that a femoral head component 82 (illustrated in FIG. 1) may be seated therein. It will be appreciated that the femoral head component 82 may articulate within the bearing cavity 147. Such articulation between the femoral head component 82 and the bearing cavity 147 of the bearing component 140 may constitute the second articulation of the bipolar prosthesis 10.

The bearing component 140 may further comprise a recess 146, which may be formed as part of the inner surface 144 of the bearing component 140. The recess 146 may be an annular recess. The recess 146 may be positioned adjacent the bearing cavity 147 on one side and may form a first junction 154 therebetween. On the opposite side, the recess 146 may also be positioned adjacent the first longitudinal wall 153e of the rim 153 and may form a second junction 155 therebetween. In other words, the recess 146 may essentially be sandwiched between the bearing cavity 147 and the first longitudinal wall 153e of the rim 153.

The recess 146 may be defined on the inner surface 144 of the bearing component 140 by several distinct surfaces. Referring specifically to FIG. 3, where the bearing component 140 is illustrated in cross section, the recess 146 may be defined by a first surface 156, a second surface 157, and a third surface 158. It is to be noted that the following description of the recess 146 is made with respect to the orientation of the bearing component 140 as illustrated in FIG. 3, such that references to upward, downward, inward, outward and the like are made with respect to that particular orientation of the bearing component 140.

Defining the recess 146 more specifically, the first surface 156 of the recess 146 may extend in a substantial laterally outward direction from the first junction 154 away from the inner surface 144. It will be appreciated that the first surface 156 may form the lowermost surface of the recess 146. The second surface 157 may extend from a terminal end 156a of the first surface 156 upwardly and inwardly in a sloping manner, or in other words the second surface 157 may taper inwardly in a proximal to distal direction with respect to the midline 152 of the bearing component 140 at an angle. It will be appreciated that the second surface 157 may comprise an angle π with respect to a line parallel to the longitudinal axis A-A of the bearing component 140 that may be between the range of about five degrees to about thirty degrees (5°-30°), specifically between about ten degrees and about twenty-five degrees (10°-25°), and more specifically between about twelve degrees to about twenty degrees (12°-20°), and even more specifically about fifteen to about sixteen degrees (15°-16°). The third surface 158 may extend in a substantial laterally inward direction from a terminal end 157a of the second surface 157. The third surface 158 may form the uppermost surface of the recess 146. It should be noted that the first and third surfaces 156 and 158, respectively, may each extend in a substantially lateral direction as defined herein.

As used herein, the phrase “substantial lateral direction” may refer to a ninety degree (90°) angle with respect to the longitudinal axis A-A of the bearing component 140, or to an angle that is greater than ninety degrees, but not more than one-hundred and thirty-five degrees (135°), or to an angle that is less than ninety degrees, but not less than forty-five degrees (45°). In other words, a “substantial lateral direction” may refer to an angle between forty-five degrees (45°) and one-hundred and thirty-five degrees (135°) with respect to the longitudinal axis A-A of the bearing component 140.

It will be appreciated that the dashed lines 156b and 158b in FIG. 3 represent the embodiment of the recess 146 that may be defined at the uppermost or lowermost surfaces by an angled surface that extends in a substantial lateral direction that is other than a substantial ninety degree angle with respect to the longitudinal axis A-A of the bearing component 140.

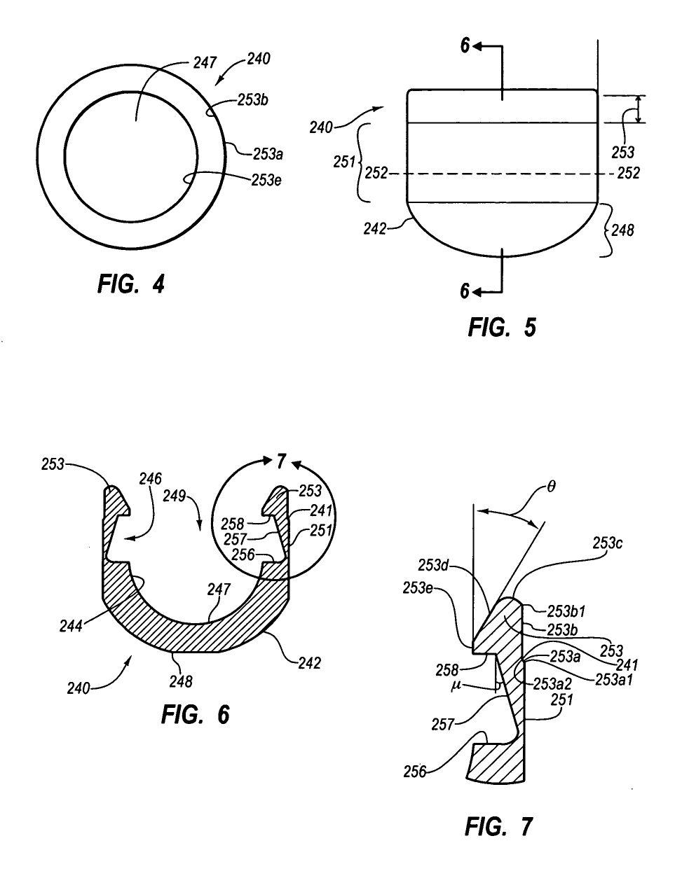

It will be appreciated that FIGS. 4-7 illustrate another embodiment of a bearing component 240, in which similar reference numerals are utilized on similar features discussed above with respect to the bearing component 140 of FIGS. 2 and 3, except that the first digit of the three digit reference numeral has been changed. For example, the bearing component of the present embodiment is identified by reference numeral 240, whereas the bearing component of the previous embodiment illustrated in FIGS. 2 and 3 is identified by reference numeral 140. For the sake of streamlining the present disclosure, only the differences between the embodiments of the bearing component will be discussed at length herein.

Referring now to FIGS. 4-7, the bearing component 240 may comprise an outer surface 242, an inner surface 244, a recess 246, a bearing cavity 247 and an opening 249 into the bearing cavity 247. In this embodiment, all references will be made with respect to the orientation of the bearing component 240 illustrated in FIGS. 4-7. It will be appreciated that the bearing component 240 may also be configured and dimensioned to seat within the interior cavity 26 of the shell component 20 and may be attached thereto by a means for attaching the bearing component 240 to the shell component 20.

The rim 253 of the bearing component 240 may be defined by five surfaces, similar to the rim 153 of the previous embodiment. However, the surfaces of the rim 253 may not be the same as the surfaces that define the previous rim 153 embodiment. As illustrated best in FIG. 7, the rim 253 of the bearing component 240 may be defined by a laterally extending wall 253a, a substantially cylindrical wall 253b, a curved or arcuate wall having a radius of curvature 253c, a tapered wall 253d, and a longitudinal wall 253e.

The laterally extending wall 253a of the rim 253 may extend inwardly from a first terminal end 253a1 of the cylindrical surface 251 toward the inner surface 244 of the bearing component 240. It will be appreciated that a ledge 241 may be formed at or near the junction of the laterally extending wall 253a and the terminal end of the cylindrical surface 251. The ledge 241 may be used as part of a means for attaching the bearing component 240 to the shell component 20. For example, the ledge 241 may mate with a corresponding ledge formed in the shell component 20 to form an interference fit, thereby maintaining the bearing component 240 within the shell component 20.

The substantially cylindrical wall 253b of the rim 253 may extend upwardly from a second terminal end 253a2 of the laterally extending wall 253a. It will be appreciated that the wall 253b may be substantially cylindrical or the wall 253b may slope without departing from the scope of the present disclosure.

The curved or arcuate wall 253c may comprise a radius of curvature and may extend from a terminal end 253b1 of the substantially cylindrical wall 253b to the tapered wall 253d. In other words, the curved wall 253c may join the substantially cylindrical wall 253b and the tapered wall 253d together.

The tapered wall 253d may extend downwardly and inwardly, or in other words may taper inwardly in a distal to proximal direction with respect to the midline 252, from the curved wall 253c. The tapered wall 253d may comprise an angle θ with respect to a line parallel to the longitudinal axis A-A of the bearing component 240 that may be between the range of about ten degrees to about fifty degrees (10°-50°), specifically between about twenty degrees and about forty degrees (20°-40°), and more specifically between about twenty-five degrees to about thirty-five degrees (25°-35°), and even more specifically about thirty degrees (30°).

Finally, the longitudinal wall 253e may be substantially the same and unchanged with respect to the longitudinal wall 153e of the previous embodiment.

It will be appreciated that the recess 246 may be substantially similar to the recess 146 of the previous bearing component 140 embodiment. For example, the recess 246 may be an annular recess, and may be comprised of three surfaces, namely a first surface 256, a second surface 257 and a third surface 258, which may be similar to surfaces 156, 157, and 158. It should be noted that the first and third surfaces 256 and 258, respectively, may each extend in a substantially lateral direction as defined herein, and may include surfaces similar to 156b and 158b without departing from the scope or spirit of the present disclosure.

The second surface 257 may comprise an angle μ that may be substantially similar to the angle π of bearing component 140, and may be between the range of about five degrees to about thirty degrees (5°-30°), specifically between about ten degrees and about twenty-five degrees (10°-25°), and more specifically between about twelve degrees to about twenty degrees (12°-20°), and even more specifically about fifteen and about sixteen degrees (15°-16°).

Referring now to FIGS. 8-13, the present disclosure comprises multiple embodiments of a free floating ring 60, which may include the same or similar features. Irrespective of the embodiment, the free floating ring 60 may be configured and dimensioned for being inserted into the recess 146 or 246 of the bearing component 140 or 240. It will be appreciated that each embodiment of the free floating ring 60 described herein may be movable within the recess 146 or 246 of the bearing component 140 or 240. The ring 60 of the present disclosure may function together with the recess 146 or 246 of the bearing component 140 or 240 to retain the femoral head component 82 within the bearing cavity 147 or 247 to form a bipolar hip implant.

For reference purposes, it will be appreciated that in FIGS. 8-13 the femoral head component 82 may be inserted into the bipolar prosthesis 10, and particularly through the ring 60, from the top of the page down in the direction of the direction arrow labeled “A” in FIGS. 8 and 12.

It will be appreciated that the rings 60 of the present disclosure may be manufactured from various biocompatible materials, including biocompatible polymeric materials such as polyethylene or ultra high molecular weight polyethylene, or other biocompatible materials that may be adapted for use as a ring as described herein, such as ceramic materials, or even biocompatible metallic materials, without departing from the scope of the present disclosure.

Referring specifically to FIGS. 8-10, one embodiment of a free floating ring is designated by reference numeral 160 and may generally comprise an inner surface 162, an outer surface 164, a top surface 166 and a bottom surface 168. The ring 160 may also comprise a gap or a slit 170, which may allow the ring 160 to expand or contract for aiding in the insertion process of the femoral head component 82. In other words, if a gap or slit 170 is present then the ring may be considered a discontinuous ring. However, it will be appreciated that the ring 160 may be a substantially circular object, whether continuous or discontinuous, and may further comprise a vacant substantially circular center 161. It will be appreciated that as used herein the phrase “substantially circular” means circular, whether a perfect circle or not, oval, ovoid, elliptical, egg shaped, or another shape that is not a perfect circular, but rather has a shape similar to those shapes described above in this definition.

The ring 160 may comprise elastic characteristics by virtue of the material from which it may be manufactured and may also comprise shape memory. Accordingly, the ring 160 may be expanded or contracted, due in part to the gap or slit 170 and the elastic material from which the ring 160 may be manufactured, when an external force is applied thereto. After the force has been released or removed from the ring 160, the ring 160 may return back to its original shape, configuration and orientation due to the elastic nature and shape memory of the ring 160.

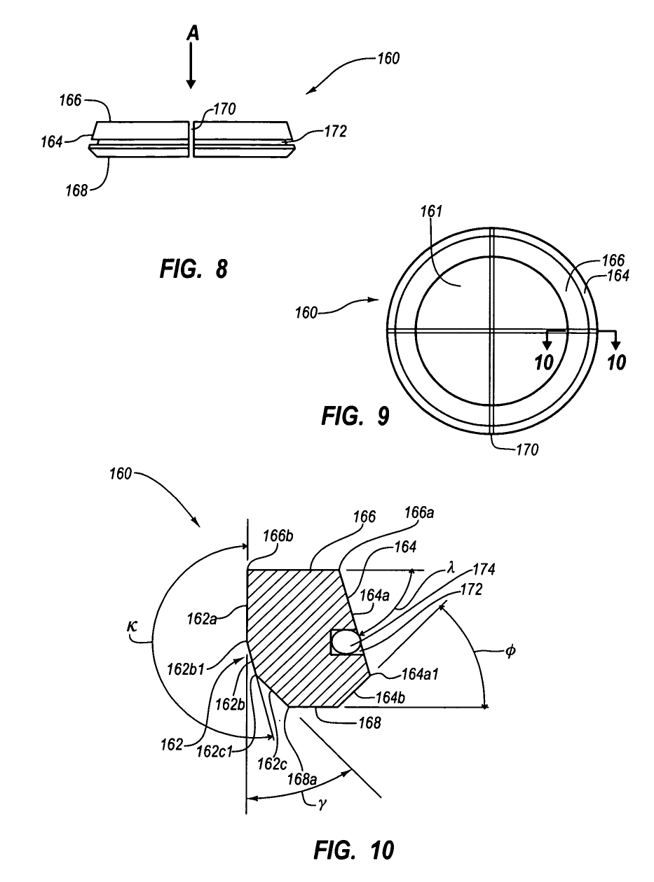

The ring 160 may comprise a recess or slot 172 that may be formed within the outer surface 164 (illustrated best in FIG. 10), and may further comprise a split ring 174 that may function as a spring. It will be appreciated that the shape of the recess or slot 172 may be any suitable shape for receiving the split ring 174 therein without departing from the spirit or scope of the present disclosure. For example, the split ring 174 may be utilized to aid the ring 160 in returning back to its original shape, configuration and orientation. The split ring 174 may be a wire made from any suitable biocompatible material for providing a spring mechanism to aid the ring 160 in returning to its original unbiased position. However, it will be appreciated that such a recess or slot 172 and split ring 174 mechanism may or may not be present in the various ring embodiments 60 disclosed herein without departing from the scope of the present disclosure.

The ring 160 of the present disclosure comprises a unique shape that aids in the insertion process of the femoral head component 82. For reference purposes, it will be appreciated that the femoral head component 82 may enter into the center 161 of the ring 160 from the top surface 166 and may move downward through the center 161 of the ring 160 during the insertion process. The shape of the ring 160 may function to aid in the insertion and retention of the femoral head component 82.

Specifically referring to FIG. 10, the ring 160 may be shaped such that the top surface 166 may be substantially parallel to the bottom surface 168. Further, the outer surface 164 may be comprised of two tapered or sloped surfaces, namely a first outer surface 164a and a second outer surface 164b, that each taper or slope in opposite directions. The inner surface 162 may be comprised of a plurality of surfaces, namely a first inner surface 162a, a second inner surface 162b, and a third inner surface 162c.

With respect to the outer surface 164, the first outer surface 164a may taper or slope from a terminal end 166a of the top surface 166 downwardly and outwardly. In other words, the first outer surface 164a may taper or slope outwardly in a distal to proximal direction with respect to the midline 152 of the bearing component 140 when the ring 160 is located in the recess 146 thereof. It will be appreciated that the taper or slope of the first outer surface 164a may comprise an angle λ with respect to the top surface 166 that may fall within a range of angles between about ninety degrees to about one-hundred twenty degrees (90°-120°), specifically between about ninety-five degrees to about one-hundred fifteen degrees (95°-115°), and more specifically about one-hundred degrees to about one-hundred ten degrees (100°-110°).

The second outer surface 164b may extend from a terminal end 164a1 of the first outer surface 164 and may taper or slope downwardly and inwardly. In other words, the second outer surface 164b may taper or slope inwardly in a distal to proximal direction with respect to the midline 152 of the bearing component 140 when the ring 160 is located in the recess 146 thereof. It will be appreciated that the taper or slope of the second outer surface 164b may comprise an angle φ with respect to the bottom surface 168 that may fall within a range of angles between about fifteen degrees to about seventy degrees (15°-70°), specifically between about twenty-five degrees to about sixty degrees (25°-60°), and more specifically about forty degrees to about forty-five degrees (40°-45°).

With respect to the inner surface 162, the third inner surface 162c may taper or slope from a terminal end 168a of the bottom surface 168 inwardly and upwardly. In other words, the third inner surface 162c may taper or slope inwardly in a proximal to distal direction with respect to the midline 152 of the bearing component 140 when the ring 160 is located in the recess 146 thereof. It will be appreciated that the taper or slope of the third inner surface 162c may comprise an angle γ with respect to the first inner surface 162a that may fall within a range of angles between about fifteen degrees to about seventy degrees (15°-70°), specifically between about twenty-five degrees to about sixty degrees (25°-60°), and more specifically about forty degrees to about forty-five degrees (40°-45°).

The second inner surface 162b of the ring 160 may taper or slope from a terminal end 162c1 inwardly and upwardly. In other words, the second inner surface 162b may taper or slope inwardly in a proximal to distal direction with respect to the midline 152 of the bearing component 140 when the ring 160 is located in the recess 146 thereof. It will be appreciated that the taper or slope of the second inner surface 162b may comprise an angle κ with respect to the first inner surface 162a that may fall within a range of angles between about one-hundred eighty degrees to about two-hundred ten degrees (180°-210°), specifically between about one-hundred ninety degrees to about two-hundred degrees (190°-200°), and more specifically about one-hundred ninety-four degrees to about one-hundred ninety-six degrees (194°-196°).

The first inner surface 162a may extend upwardly in a substantially longitudinal direction from a terminal end 162b1 of the second inner surface 162b. The first inner surface 162a may join or terminate at a second terminal end 166b of the top surface 166, thereby forming or completing the outer shape of the ring 160.

It will be appreciated that once the femoral head component 82 is inserted into the bearing cavity 147 of the bearing component 140, the femoral head component 82 may contact or bear against the second inner surface 162b of the ring 160. Further, after the femoral head component 82 has been fully installed and seated in the bearing cavity 147, the top surface 166 of the ring 160 may contact the third surface 158, which may also be the uppermost surface, of the recess 146 of the bearing component 140, thereby retaining the femoral head component 82 within the bearing cavity 147 of the bipolar prosthesis 10.

The ring 160 may be configured and dimensioned to be movable within the recess 146 of the bearing component 140. As such, the ring 160 may be smaller than the corresponding recess 146 such that a specific, limited and known amount of play between the ring 160 and the recess 146 may be present. The shape of the ring 160 and the corresponding recess 146 allow the ring 160 to move into the wider portion of the recess 146 when an external force is applied thereto, thereby allowing the ring 160 to expand. Such expansion of the ring 160 may allow the overall inner diameter of the center 161 of ring 160 to increase, thereby allowing passage of the femoral head component 82, which has a larger diameter than the ring 160 when the ring 160 is in its natural, unbiased position or orientation.

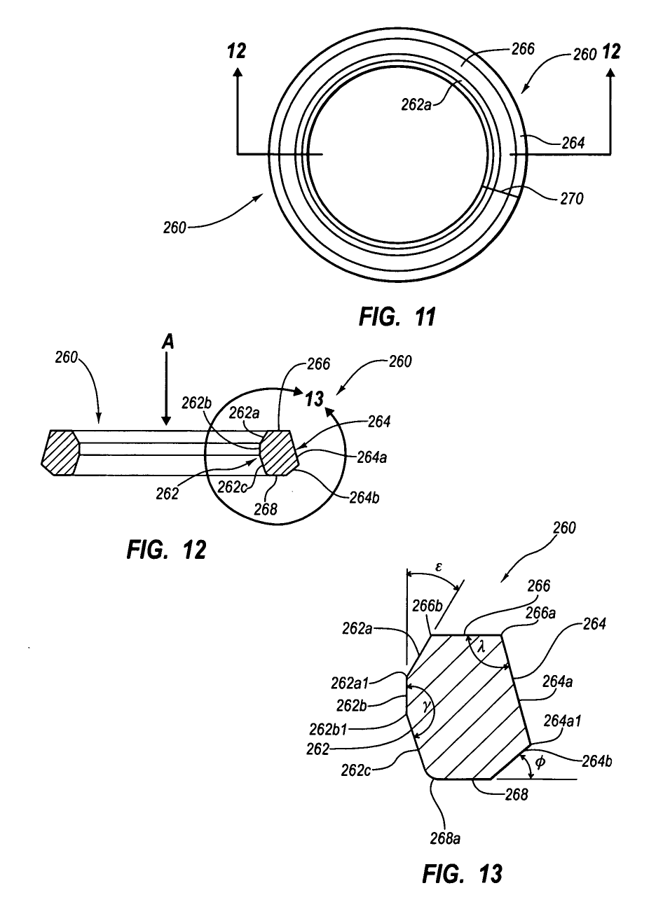

Referring now to FIGS. 11-13, an alternative embodiment of the ring is generally referred to by reference numeral 260. It is to be understood that the alternative embodiment of the ring 260 may comprise many or all of the functions and structural features of the ring 160 described above. In addition, the present embodiment of the ring 260 may comprise new functions and structural features, or modifications of the structural features, that may or may not be present in the previous embodiment of the ring 160. Accordingly, it will be appreciated that similar structural features of the rings 160 and 260 will be designated using similar reference numerals where possible, with the only exception being the first digit of the three digit reference numeral has been changed to correspond with the appropriate ring embodiment. Because the ring embodiments 160 and 260 may comprise similar structural features, only the differences will be addressed specifically herein for the sake of streamlining the disclosure.

It will be appreciated that angles λ and φ of the first and second outer surfaces 264a and 264b, respectively, of the ring 260 may be substantially the same as the corresponding surfaces of the ring 160. However, a difference between the ring 260 of the present embodiment and the ring 160, may include a change in the shape of the bottom surface 268 and the inner surface 262 of the ring 260.

Specifically, the bottom surface 268 of the ring 260 may comprise a curved or arcuate corner 268a, which has a radius of curvature. The corner 268a may further act as a junction between the bottom surface 268 and a third inner surface 262c. The corner 268a radius may provide a lead in surface along with the third inner surface 262c that may allow the femoral head 82 to more easily locate the femoral head 82 within ring 260. The corner 268a radius may also function to center the femoral head component 82 as it is lead into the inner surface 262 of the ring 260. Accordingly, the femoral head component 82 may be positioned within the ring 260 such that the femoral head component 82 may contact the third inner surface 262c and may slide down said third inner surface 262c until the femoral head component 82 reaches a position of stability.

It will be appreciated that the inner surface 262 of the ring 260 may comprise several changes with respect to the inner surface 162 of the ring 160. The first inner surface 262a may be shorter than the corresponding first inner surface 162a, and may taper or slope from a second terminal end 266b of the upper surface 266 instead of extending longitudinally from the second terminal end 166b as does the first inner surface 162a. It will be appreciated that the taper or slope of the first inner surface 262a may comprise an angle ε formed with respect to the second inner surface 262b that may fall within a range of angles between about ten degrees to about fifty degrees (10°-50°), specifically between about twenty degrees and about forty degrees (20°-40°), and more specifically between about twenty-five degrees to about thirty-five degrees (25°-35°), and even more specifically about thirty degrees (30°).

The second inner surface 262b may extend downwardly in a substantially longitudinal direction from a terminal end 262a1 of the first inner surface 262a, instead of tapering or sloping at an angle from the terminal end 162b1 as does the second inner surface 162b.

The third inner surface 262c may be elongated with respect to the third inner surface 162c and may taper or slope from a terminal end 262b1 of the second inner surface 262b. It will be appreciated that the taper or slope of the third inner surface 262c may comprise an angle γ formed with respect to the second inner surface 262b that may fall within a range of angles between about one-hundred forty degrees to about one-hundred eighty degrees (140°-180°), specifically between about one-hundred fifty degrees and about one-hundred seventy degrees (150°-170°), and more specifically between about one-hundred fifty-five degrees to about one-hundred sixty-five degrees (155°-165°).

The elongated third inner surface 262c allows for a greater potential for contact between the femoral head component 82 and the third inner surface 262c of the ring 260 when the femoral head component 82 may be fully seated and secured within the bearing component 240.

It will be understood that the inner surfaces 162 and 262 of the respective rings 160 and 260 may be straight, linear surfaces when viewed in cross-section. However, many of the surfaces may be modified to comprise other shapes beside straight, linear surfaces. Despite the foregoing, the third inner surfaces 162c and 262c of the rings 160 and 260 may be straight, linear surfaces in cross-section, and as such are, therefore, not arcuate or curved surfaces in cross-section.

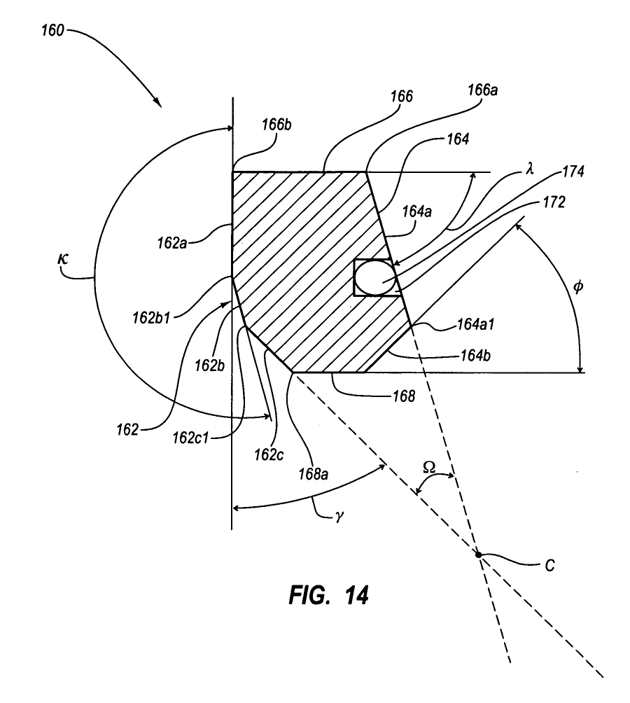

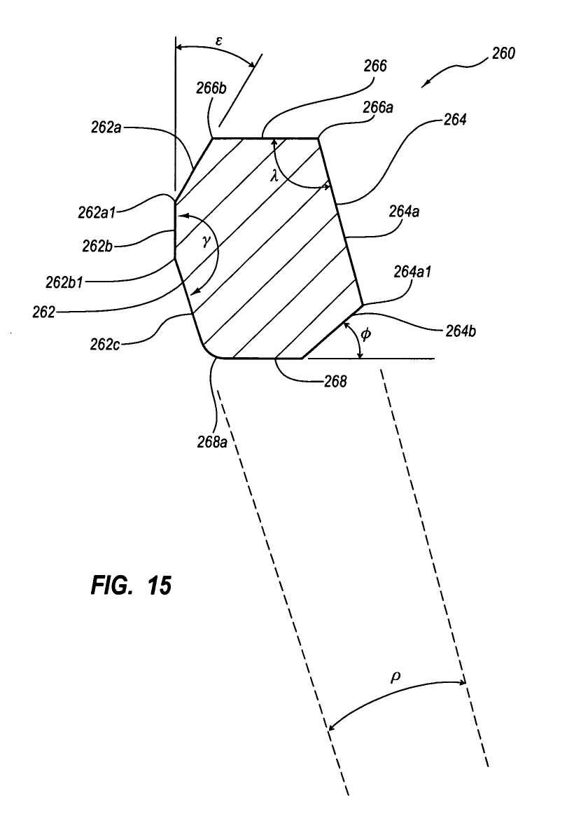

Referring now to FIGS. 14 and 15, it is to be understood that because at least a portion of the outer surface 164 or 264 and inner surface 162 or 262 of the respective rings 160 and 260 taper or slope in generally the same direction and at different angles, that there is a point of convergence “C” where the outer surface 164 or 264 may intersect the inner surface 162 or 262. Such a point of convergence “C” with respect to each of the rings 160 and 260 is illustrated in FIGS. 14 and 15. Specifically, FIG. 14 illustrates a point of convergence “C” of the first outer surface 164a and the third inner surface 162c of the ring 160. It will be appreciated that an angle Ω may be formed at the point of convergence “C,” wherein the angle Ω may fall within a range of angles between about twenty degrees to about forty degrees (20°-40°), specifically between about twenty-five degrees and about thirty-five degrees (25°-35°), and more specifically between about twenty-nine degrees and about thirty degrees (29°-30°).

It will be appreciated that the point of convergence “C” is not specifically illustrated in FIG. 15. However, as demonstrated in FIG. 15, the dashed lines extending from the first outer surface 264a and the third inner surface 262c may eventually intersect to form the point of convergence at such an intersection. It will be appreciated that an angle ρ may be formed at the point of convergence “C,” wherein the angle ρ may fall within a range of angles between about one degree to about fifteen degrees (1°-15°), specifically between about three degrees and about twelve degrees (3°-12°), and more specifically between about five degrees and about ten degrees (5°-10°).

Referring now to the method of operation of the foregoing structural components of the bipolar prosthesis 10, no matter which embodiment of the bearing component 140 or 240 and the free floating ring 160 or 260 are used, the same basic method and operation of use of the bipolar prosthesis 10 may be employed. Specifically, insertion of the femoral head component 82 into the bipolar prosthesis 10 may be accomplished by pushing the femoral head component 82 against the top surface 166 or 266 of the ring 160 or 260, thereby forcing the ring 160 or 260 up into the recess 146 or 246 of the bearing component 140 or 240. As force is applied to the top surface 166 or 266, the outside surface 164 or 264 of the ring 160 or 260 may slide against the second surface 157 or 257 of the recess 146 or 246 thereby expanding the ring 160 or 260 within the recess 146 or 246.

When expansion of the ring 160 or 260 occurs, the diameter of the center 161 or 261 of the ring 160 or 260 enlarges such that the femoral head component 82 may be allowed to pass thereby. Thereafter, the ring 160 or 260 may slide within the recess 146 or 246 until the top surface 166 or 266 comes into contact with the third surface 158 or 258 of the recess 146 or 246, thereby capturing the femoral head component 82 and allowing the femoral head component 82 to settle into a position of stability within the ring. It is to be understood that the top surface 166 or 266 of the ring 160 or 260 may abut the third surface 158 or 258 of the recess 146 or 246 thereby retaining the ring within said recess 146 or 246.

It will be appreciated that the structure and apparatus disclosed herein is merely one example of a means for attaching the bearing component to the shell component, and it should be appreciated that any structure, apparatus or system for attaching the bearing component to the shell component which performs functions the same as, or equivalent to, those disclosed herein are intended to fall within the scope of a means for attaching the bearing component to the shell component, including those structures, apparatus or systems for attaching the bearing component to the shell component which are presently known, or which may become available in the future. Anything which functions the same as, or equivalently to, a means for attaching the bearing component to the shell component falls within the scope of this element.

Those having ordinary skill in the relevant art will appreciate the advantages provide by the features of the present disclosure. For example, it is a potential feature of the present disclosure to provide a modular bipolar prosthesis which is simple in design and manufacture. Another potential feature of the present disclosure is to provide such a bipolar prosthesis having a shell component, a bearing component, and a free floating ring, wherein a straight, linear portion in cross-section of the inner surface of the ring contacts the femoral head component. It is a further potential feature of the present disclosure, in accordance with one aspect thereof, to provide a uniquely shaped free floating ring for centering the femoral head component within the inner surface of the ring. Another potential feature of the present invention is to provide an inner ring surface and an outer ring surface that may comprise a point of convergence that may fall within a certain range of angles. It will be appreciated that there may be additional potential features of the present disclosure not mentioned in this portion of the disclosure, which nevertheless may be considered pertinent to the present disclosure.

In the foregoing Detailed Description, various features of the present disclosure have been grouped together and discussed in a single embodiment, while differences between embodiments have been discussed in isolation for the purpose of streamlining the disclosure. This method of disclosure is not to be interpreted as reflecting an intention that the disclosure requires more features than are expressly recited, whether in claims or otherwise. Rather, as the following claims reflect, inventive aspects lie in less than all features of a single foregoing disclosed embodiment. Thus, the following claims are hereby incorporated into this Detailed Description of the Disclosure by this reference, with each claim standing on its own as a separate embodiment of the present disclosure.

It is to be understood that the above-described arrangements are only illustrative of the application of the principles of the present disclosure. Numerous modifications and alternative arrangements may be devised by those skilled in the art without departing from the spirit and scope of the present disclosure and the appended claims are intended to cover such modifications and arrangements. Thus, while the present disclosure has been shown in the drawings and described above with particularity and detail, it will be apparent to those of ordinary skill in the art that numerous modifications, including, but not limited to, variations in size, materials, shape, form, function and manner of operation, assembly and use may be made without departing from the principles and concepts set forth herein.

1. A bipolar prosthesis, comprising:
a shell component having an outer surface and an inner surface, wherein the inner surface defines a cavity;
a bearing component configured and dimensioned to be located and seated within the cavity of the shell component and having an outer surface and an inner surface, wherein the inner surface of the bearing component defines a bearing cavity and a recess; and
a ring configured and dimensioned for being located within the recess of the bearing component and having an outer surface and an inner surface, wherein the outer surface of the ring comprises a first tapered surface and the inner surface of the ring comprises a second tapered surface;
wherein the first tapered surface and the second tapered surface of the ring converge to form an angle that falls within a range of angles between about one degree and about fifteen-degrees.

2. (canceled)

3. The bipolar prosthesis of claim 1, wherein the shell component is a substantially semi-spherical member, and the outer surface of the shell component is configured and dimensioned to articulate with a patient's natural acetabulum, and wherein the inner surface of the shell component is dome shaped, and the cavity is semi-spherical for receiving the bearing component therein.

4. The bipolar prosthesis of claim 1, wherein the prosthesis further comprises a means for attaching the bearing component to the shell component.

5. The bipolar prosthesis of claim 1, wherein the bearing component comprises an apex region and a midline, and wherein the outer surface of said bearing component forms a substantially semi-spherical dome that extends from approximately the apex region of the bearing component to approximately the midline of the bearing component.

6. The bipolar prosthesis of claim 5, wherein the bearing component comprises a rim and an opening to the bearing cavity located at the rim, wherein the outer surface of the bearing component comprises a cylindrical surface that extends below the midline of said bearing component toward the opening of the bearing cavity and terminates at the rim of said bearing component.

7. The bipolar prosthesis of claim 6, wherein the rim of the bearing component circumferentially extends around the perimeter of the bearing component, and is defined by a first laterally extending wall, a first tapered wall, a second laterally extending wall, a second tapered wall, and a first longitudinal wall.

8. The bipolar prosthesis of claim 7, wherein the first laterally extending wall extends at substantially a ninety degree angle with respect to a longitudinal axis of the bearing component.

9. The bipolar prosthesis of claim 7, wherein the first tapered wall tapers at an angle with respect to a line parallel to a longitudinal axis of the bearing component that is between a range of about fifteen degrees to about sixty-five degrees.

10. (canceled)

11. (canceled)

12. The bipolar prosthesis of claim 7, wherein the second laterally extending wall tapers at an angle with respect to a line parallel to a longitudinal axis of the bearing component that is between a range of about ten degrees to about fifty degrees.

13. (canceled)

14. (canceled)

15. The bipolar prosthesis of claim 7, wherein the first longitudinal wall extends downwardly from a terminal end of the second tapered wall in a substantial longitudinal direction.

16. The bipolar prosthesis of claim 1, wherein the bearing cavity is semi-spherically shaped to seat a femoral head component therein and to articulate with the femoral head component.

17. The bipolar prosthesis of claim 1, wherein the recess of the bearing component is positioned between the bearing cavity on one side and a rim of the bearing component on the other.

18. The bipolar prosthesis of claim 17, wherein the recess is defined within the inner surface of the bearing component by a first surface, a second surface, and a third surface.

19. The bipolar prosthesis of claim 18, wherein the first surface of the recess extends in a substantial laterally outward direction toward the outer surface of the bearing component from the inner surface of the bearing component, wherein the second surface extends from a terminal end of the first surface in a tapered manner at an angle with respect to a line parallel to a longitudinal axis of the bearing component, wherein the angle is between the range of about five degrees to about thirty degrees, wherein the third surface extends in a substantial laterally inward direction from a terminal end of the second surface, and forms the uppermost surface of the recess.

20-26. (canceled)

27. The bipolar prosthesis of claim 1, wherein the bearing component comprises a rim that is defined by a laterally extending wall, a substantially cylindrical wall, an arcuate wall, a tapered wall, and a longitudinal wall.

28. The bipolar prosthesis of claim 27, wherein the bearing component comprises a substantially cylindrical outer surface, and wherein the laterally extending wall of the rim extends inwardly from a first terminal end of the cylindrical outer surface of the bearing component toward the inner surface of the bearing component forming a ledge at a junction of said laterally extending wall and the first terminal end of the cylindrical outer surface.

29. The bipolar prosthesis of claim 28, wherein the shell component comprises a corresponding ledge, and where the ledge of the bearing component is formed as part of a means for attaching the bearing component to the shell component, such that said ledge mates with the corresponding ledge of the shell component in an interference fit, to thereby maintain the bearing component within the shell component.

30. The bipolar prosthesis of claim 29, wherein the cylindrical wall of the rim extends upwardly from a second terminal end of the laterally extending wall, and wherein the arcuate wall comprises a radius of curvature and said arcuate wall extends from a terminal end of the substantially cylindrical wall to the tapered wall, and wherein the tapered wall slopes inwardly in a distal to proximal direction with respect to a midline of the bearing component from the curved wall, and the tapered wall slopes at an angle with respect to a line parallel to a longitudinal axis of the bearing component that is between a range of about ten degrees to about fifty degrees.

31-34. (canceled)

35. The bipolar prosthesis of claim 30, wherein the longitudinal wall extends in a substantially longitudinal direction, thereby defining the rim in conjunction with said laterally extending wall, said substantially cylindrical wall, said arcuate wall, and said tapered wall.

36. The bipolar prosthesis of claim 1, wherein the ring is a free floating ring with respect to the bearing component and shell component, and as such is movable within the recess of the bearing component.

37. The bipolar prosthesis of claim 1, wherein the ring is elastic and comprises shape memory.

38. The bipolar prosthesis of claim 37, wherein the ring is defined by the inner surface, the outer surface, and further defined by a top surface and a bottom surface, and wherein the ring comprises a slit, thereby allowing said ring to expand or contract, to thereby allow said ring to expand or contract, due in part to said slit and the elasticity of said ring, when an external force is applied thereto.

39. (canceled)

40. The bipolar prosthesis of claim 1, wherein the ring is substantially circular, and wherein the prosthesis further comprises a second split ring that acts as a spring, and wherein the ring comprises a slot formed within the outer surface, such that the second split ring is received within the slot to aid said ring in returning back to its original unbiased shape, configuration and orientation.

41. (canceled)

42. The bipolar prosthesis of claim 38, wherein the top surface of the ring is substantially parallel to the bottom surface of the ring, and the outer surface is defined by a first outer surface and a second outer surface that each taper.

43. The bipolar prosthesis of claim 42, the first outer surface tapers at an angle from a terminal end of the top surface downwardly and outwardly in a distal to proximal direction with respect to a midline of the bearing component when the ring is located in the recess of the bearing component, and wherein the angle of taper of the first outer surface is between a range of about ninety degrees to about one-hundred twenty degrees.

44-46. (canceled)

47. The bipolar prosthesis of claim 42, wherein the second outer surface extends from a terminal end of the first outer surface and tapers at an angle downwardly and inwardly in a distal to proximal direction with respect to a midline of the bearing component when the ring is located in the recess of said bearing component, wherein the taper angle is between about fifteen degrees to about seventy degrees.

48-50. (canceled)

51. The bipolar prosthesis of claim 42, wherein the inner surface is defined by a first inner surface, a second inner surface, and a third inner surface.

52. The bipolar prosthesis of claim 51, wherein the third inner surface tapers at an angle from a terminal end of the bottom surface inwardly and upwardly in a proximal to distal direction with respect to a midline of the bearing component when the ring is located in the recess of said bearing component, wherein the taper angle of the third inner surface is between about fifteen degrees to about seventy degrees.

53-55. (canceled)

56. The bipolar prosthesis of claim 51, wherein the second inner surface of the ring tapers at an angle from a terminal end of the third inner surface inwardly and upwardly in a proximal to distal direction with respect to a midline of the bearing component when the ring is located in the recess of said bearing component, wherein the taper angle of the second inner surface is between about one-hundred eighty degrees to about two-hundred ten degrees.

57-59. (canceled)

60. The bipolar prosthesis of claim 51, wherein the first inner surface extends upwardly in a substantially longitudinal direction from a terminal end of the second inner surface and terminates at a second terminal end of the top surface.

61. The bipolar prosthesis of claim 51, wherein femoral head component is inserted into the bearing cavity of the bearing component, such that the femoral head component bears against the second inner surface of the ring.

62. (canceled)

63. The bipolar prosthesis of claim 1, wherein the ring is smaller than the corresponding recess of the bearing component, such that a specific, limited and known amount of play between the ring and the recess is present.

64. The bipolar prosthesis of claim 63, wherein the ring comprises a slit and is seated within the recess, and said recess comprises a first portion and a second portion, wherein the first portion has a narrower diameter than the second portion, such that when a force is applied to said ring, said ring expands and moves into the second portion of said recess, thereby allowing passage of a femoral head component.

65. The bipolar prosthesis of claim 51, wherein the first inner surface tapers at an angle formed with respect to the second inner surface and is between about ten degrees to about fifty degrees.

66. (canceled)

67. (canceled)

68. The bipolar prosthesis of claim 65, wherein the second inner surface extends downwardly in a substantially longitudinal direction from a terminal end of the first inner surface, and wherein the third inner surface tapers at an angle from a terminal end of the second inner surface that is between about one-hundred forty degrees to about one-hundred eighty degrees.

69-71. (canceled)

72. The bipolar prosthesis of claim 1, wherein the angle of convergence is between about three degrees and about twelve degrees.

73. The bipolar prosthesis of claim 72, wherein the angle of convergence is between about five degrees and about ten degrees.

74. A bipolar prosthesis, comprising:
a shell component having an outer surface and an inner surface, wherein the inner surface defines a cavity;
a bearing component configured and dimensioned to be located and seated within the cavity of the shell component and having an outer surface and an inner surface, wherein the inner surface of the bearing component defines a bearing cavity and a recess; and
a ring configured and dimensioned for being located within the recess of the bearing component and having an outer surface and an inner surface, wherein the inner surface of the ring comprises a straight, linear cross-sectional surface that bears against a corresponding ball-shaped head component when the bipolar prosthesis is operational within a patient's joint.

75. The bipolar prosthesis of claim 74, wherein the outer surface of the ring comprises a first tapered surface and the inner surface of the ring comprises a second tapered surface, and wherein the first tapered surface and the second tapered surface of said ring converge to form an angle therebetween.

76. The bipolar prosthesis of claim 75, wherein the angle is between about twenty degrees to about forty degrees.

77. (canceled)

78. (canceled)

79. The bipolar prosthesis of claim 75, wherein the angle is between about one degree and about fifteen degrees.

80. (canceled)

81. (canceled)

82. A bipolar prosthesis, comprising:
a shell component having an outer surface and an inner surface, wherein the inner surface defines a cavity;
a bearing component configured and dimensioned to be located and seated within the cavity of the shell component and having a midline, an outer surface and an inner surface, wherein the inner surface of the bearing component defines a bearing cavity and a recess; and
a ring configured and dimensioned for being located within the recess of the bearing component and having a top surface, a bottom surface, an outer surface and an inner surface;
wherein the outer surface of the ring component comprises a first outer surface and a second outer surface that each taper in opposite directions;
wherein the inner surface of the ring comprises a first inner surface, a second inner surface, and a third inner surface;
wherein the third inner surface of the ring tapers from a terminal end of the bottom surface inwardly in a proximal to distal direction with respect to the midline of the bearing component when the ring is located in the recess of said bearing component;
wherein the second inner surface of the ring tapers from a terminal end of the third inner surface inwardly in a proximal to distal direction with respect to the midline of the bearing component when the ring is located in the recess of said bearing component;
wherein the first inner surface extends upwardly in a substantially longitudinal direction from a terminal end of the second inner surface, and said first inner surface terminates at a terminal end of the top surface, thereby forming the shape of the ring.

83. A bipolar prosthesis, comprising:
a shell component having an outer surface and an inner surface, wherein the inner surface defines a cavity;
a bearing component configured and dimensioned to be located and seated within the cavity of the shell component and having a midline, an outer surface and an inner surface, wherein the inner surface of the bearing component defines a bearing cavity and an annular recess; and
a ring configured and dimensioned for being located within the recess of the bearing component and having a top surface, a bottom surface, an outer surface and an inner surface;
wherein the bearing component further comprises a rim that circumferentially extends around a perimeter of said bearing component;
wherein the annular recess is defined by a first surface, a second surface, and a third surface;
wherein the first surface of the annular recess extends in a substantial laterally outward direction toward the outer surface of the bearing component from the inner surface of the bearing component;
wherein the second surface of the annular recess extends from a terminal end of the first surface of the annular recess in a tapered manner at an angle with respect to a line parallel to a longitudinal axis of the bearing component, wherein the angle is between the range of about five degrees to about thirty degrees;
wherein the third surface of the annular recess extends in a substantial laterally inward direction from a terminal end of the second surface of the annular recess, and forms the uppermost surface of said annular recess;
wherein the outer surface of the ring component comprises a first outer surface and a second outer surface that each taper in opposite directions;
wherein the inner surface of the ring comprises a first inner surface, a second inner surface, and a third inner surface;
wherein the third inner surface of the ring tapers from a terminal end of the bottom surface inwardly in a proximal to distal direction with respect to the midline of the bearing component when the ring is located in the recess of said bearing component;
wherein the second inner surface of the ring extends upwardly in a substantially longitudinal direction from a terminal end of the third inner surface;
wherein the first inner surface tapers from a terminal end of the second inner surface outwardly in a proximal to distal direction with respect to the midline of the bearing component when the ring is located in the recess of said bearing component, thereby forming the shape of the ring; and
wherein the taper of the first outer surface of the ring and the taper of the third inner surface of the ring converge to form an angle that falls within a range of angles between about one degree and about fifteen degrees.
